(19) United States
(12) Patent Application Publication
(10) Pub. No.: US 2014/0012137 A1
(43) Pub. Date: Jan. 9, 2014

(54) Hand-Held Dual-Magnification Dermatoscope

(71) Applicant: Robert Rosen, Marlboro, NJ (US)

(72) Inventor: Robert Rosen, Marlboro, NJ (US)

(21) Appl. No.: 13/543,709

(22) Filed: Jul. 6, 2012

(51) Int. Cl. A61B 6/00

(52) U.S. Cl. 600/476

(57) ABSTRACT

A one-piece hand-held dermatoscope have two magnifier lenses, a larger rectangular-shaped lens between two and three times magnification power, and a smaller circular lens between eight and 15 times magnification power. A polarizer is position between the lens and a patient's skin. In one embodiment, the polarizer is radial, such that cross-polarization is achieved when the light reflect off the skin and passes through it a second time. In another embodiment, the portion of the polarizer through which the light passes to the skin is linear in a first direction, and the portion through which the light passes back from the skin is linear in a second direction that is perpendicular to the first direction, enabling cross-polarization.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention relates generally to the field of dermatoscopes, and more particularly to a pocket-sized dermatoscope with two different magnification lenses, each having cross-polarization.

2. Background Art

Dermatoscopy (also known as dermoscopy or epiluminescence microscopy) refers to the use of a dermatoscope to examine the skin for aberrations or disease. Dermatoscopes were originally placed in contact with the skin through a glass plate moistened with oil or other liquid to eliminate reflections from the outer skin surface. A new generation of dermatoscopes that do not require skin contact were developed by 3Gen. One—sold under the trademark “Lumio”—has a circular 75 mm lens of 2×magnification. Forty light-emitting diodes are positioned along the periphery of the lens. There is no patent on this product, which is instead described in http://www.dermlite.com/cms/en/products/handheld-products/lumio.html. The device employs cross-polarization, but it is unclear where the two filters are placed. Two AA batteries are used to power the device. This device is useful for making quick scans of large areas of skin for a variety of skin ailments. It is a hand-held device, but is much larger than can fit in one's pocket, and has significant weight that fatigues a doctor using it for long periods of time.

A second device by 3Gen is a dermoscopy epilumination device having 10×magnification and cross polarization, which is described in various alternate embodiments in U.S. Pat. Nos. 7,006,223, 7,027,253, 7,167,243 and 7,167,244. These embodiments have a circular lens around which a plurality of light-emitting diodes are positioned to radiate light onto and through the surface of the skin, which is reflected back through the lens and polarizing filters. One set of alternating diodes has two filters that polarize the light in parallel, and another set of diodes has two filters that cross-polarize the light. One version employs different colored diodes. In practice, dermatologists seldom use the parallel-polarization feature, which allows too much light reflected from the skin's surface to transmit through the lens, thereby obscuring the light that penetrates through the skin and reflects off the aberration being examined.

This device is useful for conducting a more detailed examination of the a mole or other skin aberration than the Lumio, but takes considerably longer time to use. It is far more compact and lighter than the Lumio, and can fit in one's pocket. Either primary or rechargeable lithium batteries are used to power the diodes.

When using these devices, a dermatologist would have to switch between the devices once the cursory screening using the Lumio showed an aberration of interest. This slows down the examination, and is inconvenient for the doctor.

It would be advantageous to have a single light-weight pocket-sized dermatoscope that provided both functions, having two lens of roughly 2× and 10×magnification. It would also be advantageous to have a dermatoscope that uses a simplified light source that illuminates both lenses without complex rings of LEDs encircling each lens.

### BRIEF SUMMARY OF THE INVENTION

The present invention solves these problems by creating a single unit magnifier comprising two magnifiers, a large one at 2×magnification, and a small one at 10×magnification. In the preferred embodiment, the large magnifier is rectangular, having a length of 97 mm, and a height of 23 mm. This presents a smaller magnifier than the Lumio but has a greater field of view because the eye naturally moves from side to side, not vertically. This avoids having a considerable waste of space in the vertical dimension for circular lenses. The rectangular shape thereby increases the viewable skin while decreasing the size and weight of the device.

The smaller, higher-powered magnifier (10×) is centered in the longer axis on the longitudinal side of the larger magnifier and is circular in shape to enable the physician to observe in greater detail a skin aberration. It need not be rectangular because in the preferred embodiment, its diameter is substantially less than the shorter dimension of the rectangular lens.

The device is lit by a chip-on-board (COB) light-emitting diode, a thin strip that is preferably placed along the side of the larger rectangular lens opposite the smaller and higher-powered magnifier. A thin polarizing filter covers both lenses positioned between the lenses and the skin. The light is emitted through this layer, then reflects back from the skin through it a second time, either through a single circularly polarized filter or through two perpendicularly oriented linear polarizing filters. Either method creates cross-polarization, substantially eliminating glare from light that reflects off the surface of the skin. The linear array of LED lights allows a simplified single polarization film to be employed. The linear array is also less expensive and easier to make than using a ring of individual LEDs that encircle either lens—as in the prior-art dermatoscope—and it illuminates both lenses.

A rechargeable lithium iron phosphate battery (LiFePO₄) is used to store the energy. It has greater chemical stability than some lithium-based batteries, which on rare occasions can catch of fire during recharging. It also provides a light-weight efficient high-energy battery source that holds a charge longer than standard lithium batteries. Compared to the Lumio device, which uses AA batteries, the LiFePO₄ battery provides substantially greater energy for its size and weight, enabling a more portable unit having a long-lasting power source.

A physician may use this dermatoscope to perform a relatively quick examination of a patient's skin by looking through the lower-powered magnifier to detect any aberrations of potential interest, and then perform a more thorough examination using the high-powered magnifier for any such aberration. The physician can perform both functions using a single portable hand-held device without the need to take extra time to exchange one dermatoscope for another, and without the risk of not finding the aberration when exchanging dermatoscopes.

### BRIEF DESCRIPTION OF THE SEVERAL VIEWS OF THE DRAWINGS

The accompanying drawings illustrate the invention, where like reference numerals indicate the same feature throughout the drawings:

FIG. 1 shows a top view of the dermatoscope of the preferred embodiment of the present invention without the top housing; and

FIG. 2 shows a 3-dimensional exploded view of the components of the dermatoscope shown in FIG. 1.

### DETAILED DESCRIPTION OF THE INVENTION

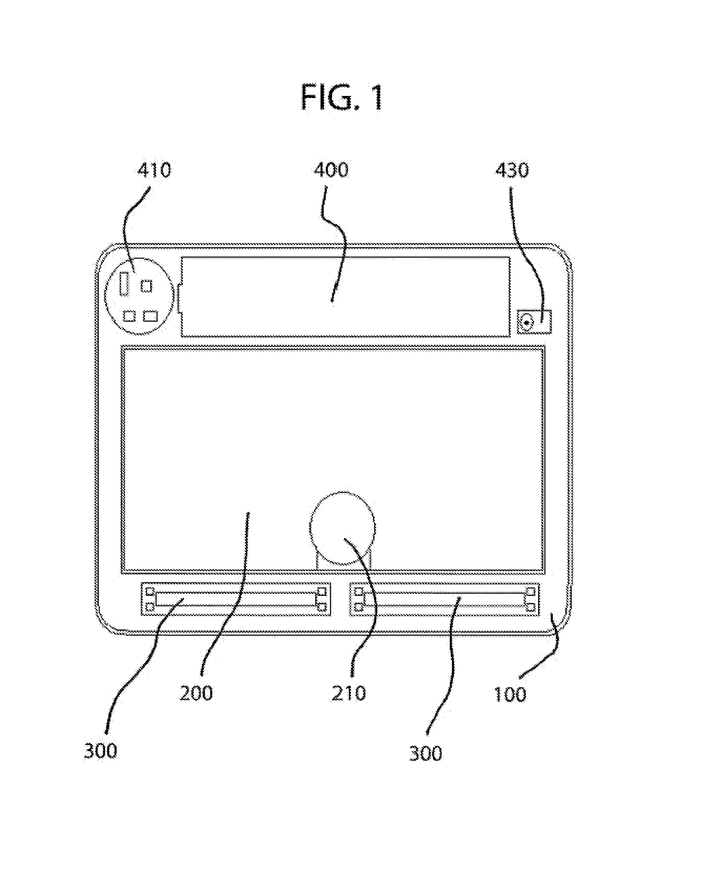

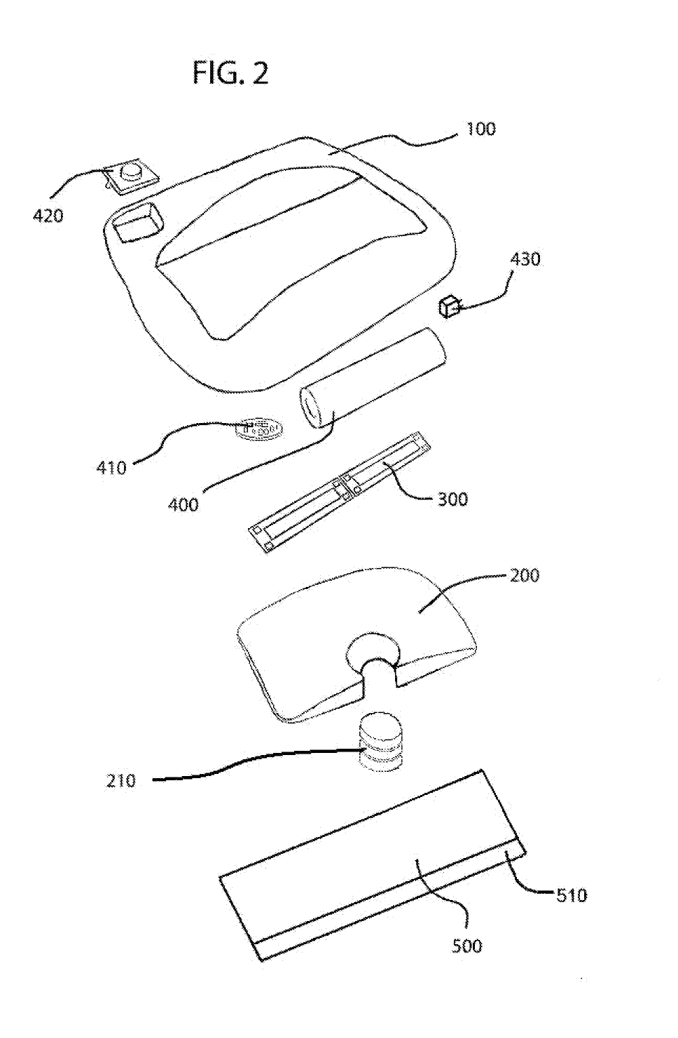

The drawings are for illustrative purposes of the preferred embodiment of the present invention. FIGS. 1 and 2 show the various components of the preferred embodiment. Top housing 100 comprises the housing for the invention, into which the various components are placed. It is preferably made of plastic.

The dermatoscope is powered by battery 400, which is preferably a rechargeable lithium iron phosphate battery (LiFePO₄). This battery has the advantage of storing considerable electrical charge at 3 amps at about 3.3 volts, yet is very stable, unlike standard lithium batteries than can catch fire when mishandled or damaged. The cost and complexity of the product is greatly reduced by avoiding such circuitry, such as that found in cell phones.

Charging jack 430 is a standard charging jack that interrupts switching circuit 410's connection to battery 400 to enable recharging. Charging jack 430 is well known to those in the art.

Opposite battery 400 is a wall on which light-emitting diodes 300 are positioned. Light-emitting diodes 300 are preferably a chip-on-board (COB) light-emitting diode, which typically is manufactured in a thin strip, as shown in light-emitting diode 300 in FIG. 2. These thin strips expand substantially all of the longitudinal length of lower-magnification lens 200.

The lenses—lower-magnification lens 200 and greater-magnification lens 210—are shown in FIGS. 1-2. Lower-magnification lens 200 is between two and three times magnification—preferably two time magnification—and is generally rectangular in shape. Its length is 97 mm, and its width is 50 mm. At one edge along the longitudinal axis of lower-magnification lens 200 is a circular cut-out into which greater-magnification lens 210 is permanently inserted. The cut-out of lower-magnification lens 200 is centered along the longitudinal axis, and on the side of lower-magnification lens 200 that is opposite battery 400 and adjacent to light-emitting diodes 300.

Greater-magnification lens 210 is between eight and 15 times magnification power—preferably 10 times magnification—and is circular in shape. It is comprised of three layers in a Hastings Triplet configuration, a design well known in the art.

The lower edge of lower-magnification lens 200 and higher-magnification lens 210 is lined with polarizer 500, 510—a thin sheet that is may be radial or linear filter. For the circular polarizer, polarizer 500, 510 is a single uniform film. Because the light from light-emitting diodes 300 passes through this filter twice, once on the way to the skin, and a second time from the light reflected from the skin, polarizer 500 provides cross polarization as the light that reflects off the skin has its radial direction reversed relative to the radial direction from the light passing through polarizer 500 on the way towards the skin. Alternately, a linear polarizing filter strip is placed under the COB light-emitting diodes 300 and a perpendicularly oriented linear polarizing filter is placed under the lenses. For the linear polarizer, polarizer portion 510 (through which the light passes from the light-emitting diodes 300 toward the skin) is linear in a first direction, and polarizer portion 500 (through which the light passes when reflect off the skin towards magnifiers 200, 210) is linear in a second direction that is perpendicular to the first direction. Either configuration produces cross polarization that substantially eliminates glare caused by light reflecting directly off the skin's surface, which otherwise drowns out the light that penetrates through the skin and reflects off a particular feature or aberration beneath the skin's surface.

The various components are held together within top housing 100 by standard adhesive and small screws. Wires (not shown) connect both sides of battery 400 (the positive and negative sides) to light-emitting diodes 300.

The process by which the dermatoscope functions is that switch 420 is activated, connecting through wires the power stored in the batteries to the circuit board 410. Circuit board 410 ensures that a constant current is delivered to light-emitting diodes 300 as long as there is a minimum charge remaining in battery 400. Circuitry such as circuit board 410 is well known in the art, and is activated by on-off switch 420.

Once light-emitting diode 300 is activated, the light generated passes through polarizer portion 510. In one embodiment, the light is then radially polarized in one axial direction (i.e., either clockwise or counter-clockwise), and is transmitted towards the skin. In another embodiment, the light is linearly polarized in one direction, and is transmitted towards the skin. The dermatoscope is held about three inches from the skin for lower-magnification lens 200, and within an inch for greater-magnification lens 210.

Some of the light reflects off the skin's surface, and some penetrates beneath the surface to objects in various skin layers, such as an aberration, which could be a benign mole or other matter, or a malignant tumor. The light then reflects off any such object and is transmitted back. In the first embodiment, when the light passes through polarizer portion 500, the radial direction reversed, creating cross-polarization. In the second embodiment, when the light passes through polarizer portion 500 the linear direction is perpendicular to the direction of polarizer portion 510, thereby creating cross-polarization. The light then passes through lenses 200 and 210. Other light colors and wavelengths may be added. An additional switch to turn on or off the lights may also be added.

The method of using the dual-magnification dermatoscope is to activate on-off switch 420 to activate circuit board 410, which transmits electricity from battery 400 to light-emitting diode 300. The patient's skin is then scanned relatively quickly using low-magnification lens 200 held about three inches from the skin until an aberration of interest is detected.

The aberration of interest is then examined under higher-powered magnification of greater-magnification lens 210, which is held within an inch of the skin. If the aberration appears of concern, it may be removed or biopsied, immediately, or after the end of the screening examination. Once the examination continues, typically without any need for surgery, the physician moves the dermatoscope back to three inches away from the skin and uses low-magnification lens 200 to scan for aberrations. This process is repeated until all of the patient's skin has been examined.

Various other modifications may be made to that depicted in the various drawings of the preferred embodiment of the present invention without departing from the spirit and scope of the invention. Accordingly, the invention is not to be limited by the preferred embodiment shown in the various drawings and described herein, but by the scope of the claims.

1. A one-piece hand-held dermatoscope for examining skin comprising:
a first magnifier having a first magnification;
a light source for generating light positioned adjacent to at least one side of the first magnifier; and
a polarizer positioned between the first magnifier and a patient's skin;
wherein the light generated by the light source travels through the first magnifier, then through the polarizer, to the skin, where it is reflected back through the polarizer then passes through the first magnifier.

2. The one-piece hand-held dermatoscope of claim 1 in which the polarizer is a circular polarizer such that the light that passes through the circular polarizer down towards the skin is polarized in one radial direction, and the light that reflects back from the skin to the circular polarizer is polarized in the opposite radial direction, thereby creating cross polarization of the light.

3. The one-piece hand-held dermatoscope of claim 1 in which the polarizer has two portions, a first portion through which the light passes from the light source, and a second portion where the light passes when reflected back of the skin, wherein the first portion is polarized in one linear direction and the second portion is polarized in a linear direction that is perpendicular to the linear direction of the first portion, thereby creating cross-polarization.

4. The one-piece hand-held dermatoscope of claim 1 in which the light source is a chip-on-board (COB) light-emitting diode.

5. The one-piece hand-held dermatoscope of claim 1 in which the light source is powered by a rechargeable lithium iron phosphate battery.

6. The one-piece hand-held dermatoscope of claim 1 in which the first magnifier is generally rectangular in shape, having a length of no greater than 100 mm, and a width no greater than 50 mm.

7. The one-piece hand-held dermatoscope of claim 1 in which the first magnifier is between two and three times magnification power.

8. The one-piece hand-held dermatoscope of claim 7 in which the first magnifier is between two and three times magnification power, and the second magnifier is between eight and 15 times magnification power.

9. The one-piece hand-held dermatoscope of claim 8 in which the first magnifier is generally rectangular in shape, having a length of no greater than 100 mm along a longitudinal axis, and a width no greater than 50 mm, with a generally circular portion cut out at the center of the longitudinal axis adjacent to the light source, and into which the second magnifier is permanently inserted.

10. A process for examining skin using a single hand-held dermatoscope comprising the steps of:
generating light adjacent to a first magnifier;
transmitting the light through a polarizer;
transmitting the light onto and beneath a layer of skin;
reflecting the light from the layer of skin back through the polarizer;
transmitting some of the light back through the first magnifier; and
transmitting some of the light back through a second magnifier of greater magnification than the first magnifier.

11. The process for examining skin using a single hand-held dermatoscope of claim 10 in which the polarizer is a radial polarizer such that the light that is transmitting through the radial polarizer is polarized in one radial direction, and the light that is reflected back from the layer of skin and through the radial polarizer is filtered in the opposite radial direction, thereby creating cross polarization of the light.

12. The process for examining skin using a single hand-held dermatoscope of claim 10 in which the polarizer in the step of transmitting the light through a polarizer is linear, and wherein the polarizer in the step of reflecting the light from the layer of skin back through the polarizer is linear in a direction that is perpendicular to the direction of the polarizer in the step of transmitting the light through a polarizer such that cross-polarization is obtained. is a radial polarizer such that the light that is transmitting through the radial polarizer is polarized in one radial direction, and the light that is reflected back from the skin and through the radial polarizer has rotated in the opposite radial direction, thereby creating cross polarization of the light.

13. The process for examining skin using a single hand-held dermatoscope of claim 10 in which the size of first magnifier is between two and three times magnification power, and the second magnifier is between eight and 15 times magnification power.

14. The process for examining skin using a single hand-held dermatoscope of claim 13 in which a longitudinal axis of the first magnifier is no greater than 100 mm, and the width of the first magnifier is no greater than 50 mm.

15. The process for examining skin using a single hand-held dermatoscope of claim 14 in which the light source is a chip-on-board (COB) light-emitting diode.

16. The process for examining skin using a single hand-held dermatoscope of claim 15 in which the light source is powered by a rechargeable lithium iron phosphate battery.

17. A method of using a hand-held dermatoscope comprising the steps of: activating a power source to generate light in the dermatoscope; placing the dermatoscope within a few inches of a patient's skin; looking through a relatively large and low-powered magnification lens within the dermatoscope to perform a quick examination of the patient's skin until an aberration of potential concern is observed; and looking through a relatively small and high-powered magnification lens within the dermatoscope to perform a more thorough examination of the aberration to determine whether it requires removal.

18. The method of using a hand-held dermatoscope of claim 17 in which light transmitted to the layer of skin passes through a radial polarizer towards and onto the layer of skin, and is then reflected back through the radial polarizer and then the lens such that the light that passes through the radial polarizer is polarized in opposite radial directions each time it passes through the radial polarizer, thereby resulting in cross polarization.

19. The method of using a hand-held dermatoscope of claim 17 in which the polarizer has two portions, a first portion through which the light passes from the light source, and a second portion where the light passes when reflected back of the skin, wherein the first portion is polarized in one linear direction and the second portion is polarized in a linear direction that is perpendicular to the linear direction of the first portion, thereby creating cross-polarization.

20. The method of using a hand-held dermatoscope of claim 17 in which the relatively large and low-powered magnifier is between two to three times magnification, and the relatively small and high-powered magnifier is between eight and 15 times magnification.
